(19) United States
(12) Patent Application Publication
(10) Pub. No.: US 2023/0327203 A1
(43) Pub. Date: Oct. 12, 2023

(54) LITHIUM SECONDARY BATTERY

(71) Applicant: SAMSUNG SDI CO., LTD., Yongin-si, Gyeonggi-do (KR)

(72) Inventors: Sanghyung KIM, Yongin-si (KR); Sanghoon KIM, Yongin-si (KR); Myungheui WOO, Yongin-si (KR)

(21) Appl. No.: 18/044,360

(22) PCT Filed: Oct. 7, 2021
(86) PCT No.: PCT/KR2021/013789

§ 371 Date: Mar. 7, 2023

(30) Foreign Application Priority Data
Nov. 6, 2020 (KR) 10-2020-0148016

(51) Int. Cl. H01M 10/0567; H01M 10/0525; H01M 10/0569; H01M 4/583; H01M 4/38; H01M 4/36; C07D 333/48

(52) U.S. Cl. CPC H01M 10/0567; H01M 10/0525; H01M 10/0569; H01M 4/583; H01M 4/386; H01M 4/362; C07D 333/48; H01M 2004/027

(57) ABSTRACT

Provided is a lithium secondary battery, which comprises: an electrolyte comprising a non-aqueous organic solvent, a lithium salt, and an additive represented by Chemical Formula 1; a cathode comprising a cathode active material including a Si-carbon composite; and an anode comprising an anode active material.

### CROSS-REFERENCE TO RELATED APPLICATIONS

This application is a U.S. National Phase Patent Application of International Application Number PCT/KR2021/013789, filed on Oct. 7, 2021, which claims priority of Korean Patent Application Number 10-2020-0148016, filed on Nov. 6, 2020, the entire content of each of which is incorporated herein by reference.

### TECHNICAL FIELD

It relates to a lithium secondary battery.

### BACKGROUND ART

Lithium secondary batteries are attracting attention as power sources for various electronic devices because of high discharge voltage and high energy density.

As for positive active materials of lithium secondary batteries, a lithium-transition metal oxide having a structure capable of intercalating lithium ions such as LiCoO₂, LiMn₂O₄, LiNi₁₋ₓCoₓO₂ (0<x<1), and the like has been used.

As for negative active materials, various carbon-based materials such as artificial graphite, natural graphite, and hard carbon capable of intercalating and deintercalating lithium ions have been used. As electrolytes for a lithium secondary battery, organic solvents in which lithium salts are dissolved has been used.

### Technical Problem

One embodiment provides a lithium secondary battery exhibiting improved high capacity and improved cycle-life characteristics.

### Technical Solution

According to one embodiment, a lithium secondary battery including an electrolyte including a non-aqueous organic solvent, a lithium salt, and an additive represented by Chemical Formula 1, a negative electrode including a negative active material including a Si-carbon composite, and a positive electrode including a positive active material.

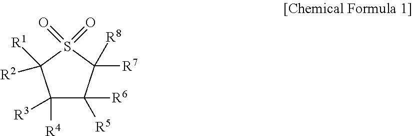

(In Chemical Formula 1,
R¹ to R⁸ are each independently a hydrogen atom, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C3 to C30 cycloalkynyl group, or a substituted or unsubstituted C6 to C30 aryl group.)

In Chemical Formula 1, the R¹ to R⁸ may each independently be a hydrogen atom, a substituted or unsubstituted C1 to C10 alkyl group, a substituted or unsubstituted C2 to C10 alkenyl group, a substituted or unsubstituted C2 to C10 alkynyl group, a substituted or unsubstituted C3 to C10 cycloalkyl group, a substituted or unsubstituted C3 to C10 cycloalkenyl group, a substituted or unsubstituted C3 to C10 cycloalkynyl group, or a substituted or unsubstituted C6 to C10 aryl group.

In one embodiment, the additive represented by Chemical Formula 1 may be sulfolane, methylsulfolane, dimethylsulfolane, or combinations thereof.

An amount of the additive represented by Chemical Formula 1 may be 0.1 wt % to 10 wt %, when the amounts of the non-aqueous organic solvent and the lithium salt are to be 100 wt %.

An amount of the Si—C carbon composite may be 0.1 wt % to 5 wt % based on the total weight of the negative active material. Furthermore, the negative active material may further include crystalline carbon.

The non-aqueous organic solvent may include a propionate-based solvent. The propionate-based solvent may be methyl propionate, ethyl propionate, propyl propionate, or combinations thereof. In addition, an amount of the propionate-based solvent may be 5 volume % to 80 volume % based on the total volume of the non-aqueous organic solvent.

The Si-carbon composite may include Si nanoparticles and amorphous carbon. According to one embodiment, the Si-carbon composite may include a core and a coating layer surrounded on the core, and the core may include amorphous carbon or crystalline carbon, and Si nanoparticles, and the coating layer may include amorphous carbon.

In one embodiment, the coating layer may have a thickness of 1 nm to 100 nm. In one embodiment, an amount of the Si nanoparticles may be 1 wt % to 60 wt % based on the total weight of the Si-carbon composite.

Other embodiments are included in the following detailed description.

### Advantageous Effects

A lithium secondary battery according to one embodiment of the present invention includes an electrolyte having good resistance-oxidation stability, and thus, the high-voltage characteristics may be improved, and in addition, may reduce resistance, thereby exhibiting high-capacity and excellent cycle-life characteristics.

### BRIEF DESCRIPTION OF THE DRAWINGS

FIG. 1 is a schematic view of a lithium secondary battery according to an embodiment.

FIG. 2 is a graph showing initial DC resistance, DC resistance at high temperature storage, and a resistance increase rate of the lithium secondary cells according to Examples 2 and 5, and Comparative Example 3.

FIG. 3 is a graph showing initial DC resistance, DC resistance at high temperature storage, and a resistance increase rate of the lithium secondary cells according to Examples 1 to 6, Reference Examples 1 and 2, and Comparative Examples 1 to 7.

FIG. 4 is a graph showing initial DC resistance, DC resistance at high temperature storage, and a resistance increase rate of the lithium secondary cells according to Examples 1 to 3, Reference Example 1, and Comparative Example 5.

### MODE FOR INVENTION

Hereinafter, embodiments of the present invention are described in detail. However, these embodiments are exemplary, the present invention is not limited thereto and the present invention is defined by the scope of claims.

In the specification, when a definition is not otherwise provided, the term ‘substituted’ refers to one in which hydrogen of a compound is substituted with a substituent selected from a halogen atom (F, Br, Cl, or I), a hydroxy group, an alkoxy group, a nitro group, a cyano group, an amino group, an azido group, an amidino group, a hydrazine group, a hydrazono group, a carbonyl group, a carbamyl group, a thiol group, an ester group, a carboxyl group or a salt thereof, a sulfonic acid group or a salt thereof, phosphoric acid or a salt thereof, a C1 to C20 alkyl group, a C2 to C20 alkenyl group, a C2 to C20 alkynyl group, a C6 to C30 aryl group, a C7 to C30 arylalkyl group, a C1 to C4 alkoxy group, a C1 to C20 heteroalkyl group, a C3 to C20 heteroarylalkyl group, a C3 to C30 cycloalkyl group, a C3 to C15 cycloalkenyl group, a C6 to C15 cycloalkynyl group, a C2 to C20 heterocycloalkyl group, or a combination thereof.

One embodiment provides a lithium secondary battery including an electrolyte including a non-aqueous organic solvent, a lithium salt, and an additive represented by Chemical Formula 1, a negative electrode including a negative active material, and a positive electrode including a positive active material.

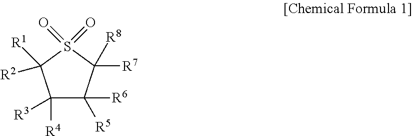

In Chemical Formula 1,
R¹ to R⁸ are each independently a hydrogen atom, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C3 to C30 cycloalkynyl group, or a substituted or unsubstituted C6 to C30 aryl group.

In one embodiment, the R¹ to R⁸ may each independently be a hydrogen atom, a substituted or unsubstituted C1 to C10 alkyl group, a substituted or unsubstituted C2 to C10 alkenyl group, a substituted or unsubstituted C2 to C10 alkynyl group, a substituted or unsubstituted C3 to C10 cycloalkyl group, a substituted or unsubstituted C3 to C10 cycloalkenyl group, a substituted or unsubstituted C3 to C10 cycloalkynyl group, or a substituted or unsubstituted C6 to C10 aryl group.

For example, the additive represented by Chemical Formula 1 may be sulfolane, methylsulfolane, for example, 3-methylsulfolane, dimethylsulfolane, for example, 2,4-dimethylsulfolane, or combinations thereof.

Herein, an amount of the additive represented by Chemical Formula 1 may be 0.1 wt % to 10 wt % based on the weight of the non-aqueous organic solvent and the lithium salt, that is, the amounts of the non-aqueous organic solvent and the lithium salt to be 100 wt % (based on the total, 100 wt % of the non-aqueous organic solvent and the lithium salt), and according to one embodiment, may be 0.5 wt % to 7.5 wt %, and according to another embodiment, 2.5 wt % to 7.5 wt %. When the amount of the additive represented by Chemical Formula 1 is satisfied in the range, the high-temperature reliability characteristics, for example, the decrease in high temperature resistance, may be realized.

The negative active material may further include crystalline carbon, together with the Si—C composite. Herein, an amount of the Si—C composite may be 0.1 wt % to 5 wt % based on the total weight, that is, a total of 100 wt %, of the negative active material.

When the negative active material including the Si—C composite is used in a battery with the electrolyte including the additive of Chemical Formula 1, the increase in resistance at high temperature may be effectively suppressed, and such effects may be largely obtained when the Si—C composite is used at 0.1 wt % to 5 wt %, and according to one embodiment, 1 wt % to 5 wt %, or another embodiment, 2.5 wt % to 5 wt %. In case of including the Si—C composite of 0.1 wt % to 5 wt % as the negative active material, the desired high-capacity and the volume expansion suppress effects may be more effectively obtained.

The Si-carbon composite may include Si nanoparticles and amorphous carbon. According to one embodiment, the Si-carbon composite may include a core and a coating layer surrounded on the core, and the core may include amorphous carbon or crystalline carbon, and Si nanoparticles, and the coating layer may include amorphous carbon.

The amorphous carbon may be soft carbon, hard carbon, mesophase pitch carbide, a sintered coke, or a mixture thereof. The crystalline carbon may be natural graphite, artificial graphite, or combinations thereof.

When the Si-carbon composite includes Si nanoparticles and amorphous carbon, a mixing ratio of the Si nanoparticles and amorphous carbon may be 2:1 to 1.5:1 by weight ratio. In addition, if the Si-carbon composite includes the core and the coating layer, an amount of the coating layer may be 0.08:1 to 0.2:1 based on the total 100 wt % of the composite, an amount of the Si nanoparticles may be 1 wt % to 60 wt % based on the total 100 wt % of the Si-carbon composite, and according to one embodiment, may be 3 wt % to 60 wt %. Furthermore, an amount of amorphous carbon or crystalline carbon included in the core may be 20 wt % to 60 wt % based on the total 100 wt % of the composite.

In addition, the coating layer may have a thickness of 1 nm to 100 nm, for example, 5 nm to 100 nm.

In addition, regardless of having the Si-carbon composite with any shape, the Si nanoparticles may have a particle diameter of 5 nm to 150 nm. For example, it may be 10 nm to 150 nm, specifically, 30 nm to 150 nm, more specifically, 50 nm to 150 nm, narrowly, 60 nm to 100 nm, and more narrowly, 80 nm to 100 nm. In the specification, a size may be a particle diameter, and may be an average particle diameter of particle diameters. In this case, the average particle diameter may mean a particle diameter (D50) measured as a cumulative volume. When a definition is not otherwise provided, an average particle diameter indicates an average particle diameter (D50) where a cumulative volume is about 50 volume % in a particle distribution. D50 may be measured by a method that is well known to those skilled in the art, for example, by a particle size analyzer, or by a transmission electron microscopic image, or a scanning electron microscopic image. Alternatively, a dynamic light-scattering measurement device is used to perform a data analysis, and the number of particles is counted for each particle size range. From this, the average particle diameter (D50) value may be easily obtained through a calculation.

In the electrolyte according to one embodiment, the non-aqueous organic solvent may include a carbonate-based solvent, and may further include a propionate-based solvent.

In the non-aqueous organic solvent, an amount of the propionate-based solvent may be 5 volume % to 80 volume % based on the total volume of the non-aqueous organic solvent. When the non-aqueous organic solvent includes the propionate-based solvent, particularly in the above amount, the gas generation at high-temperature storage or used at a high temperature, may be more effectively suppressed, particularly, in a pouch-type.

The propionate-based solvent may be methyl propionate, ethyl propionate, propyl propionate, or combinations thereof. When the propionate-based solvent is used in a mixture, the mixing ratio may be suitably controlled. For example, the propionate-based solvent may be used by mixing ethyl propionate and propyl propionate. Herein, the non-aqueous organic solvent may include ethyl propionate at 5 volume % to 40 volume %, propyl propionate at 55 volume % to 75 volume %, and the carbonate-based solvent as a residual. The mixing ratio of ethyl propionate and propyl propionate may be 25:75 to 30:70 by volume ratio. When the propionate-based solvent, ethyl propionate, and propyl propionate are used, particularly at the aforementioned amounts, the generation of gas may be more effectively suppressed, and the low temperature cycle-life characteristics may be more improved.

The carbonate-based solvent may be dimethyl carbonate (DMC), diethyl carbonate (DEC), dipropyl carbonate (DPC), methylpropyl carbonate (MPC), ethylpropyl carbonate (EPC), methylethyl carbonate (MEC), ethylene carbonate (EC), propylene carbonate (PC), butylene carbonate (BC), or combinations thereof. When the carbonate-based solvent is used in a mixture, the mixing ratio may be suitably controlled. Furthermore, the carbonate-based solvent may desirably include a mixture with a cyclic carbonate and a linear carbonate. Herein, the cyclic carbonate and the linear carbonate are mixed together in a volume ratio of about 1:1 to about 1:9, and when the mixture is used as an electrolyte, it may have enhanced performance.

In one embodiment, the non-aqueous organic solvent may further include an ester-based, ether-based, ketone-based, alcohol-based, or aprotic solvent.

The ester-based solvent may be methyl acetate, ethyl acetate, n-propyl acetate, t-butyl acetate, methyl propionate, ethyl propionate, propyl propionate, γ-butyrolactone, decanolide, valerolactone, mevalonolactone, caprolactone, and the like.

The ether-based solvent may be dibutyl ether, tetraglyme, diglyme, dimethoxyethane, 2-methyltetrahydrofuran, tetrahydrofuran, and the like, and the ketone-based solvent may be cyclohexanone and the like.

The alcohol-based solvent may include ethyl alcohol, isopropyl alcohol, and the like, and the aprotic solvent may include nitriles such as R—CN (where R is a C2 to C20 linear, branched, or cyclic hydrocarbon, and may include a double bond, an aromatic ring, or an ether bond), amides such as dimethylformamide, dioxolanes such as 1,3-dioxolane, and the like.

In addition, the organic solvent may further include an aromatic hydrocarbon-based solvent. The aromatic hydrocarbon-based organic solvent may be an aromatic hydrocarbon-based compound represented by Chemical Formula 2.

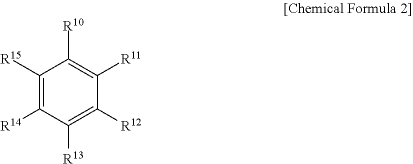

(In Chemical Formula 2, R¹⁰ to R¹⁵ are the same or different, and are selected from hydrogen, a halogen, a C1 to C10 alkyl group, a haloalkyl group, and a combination thereof.)

Specific examples of the aromatic hydrocarbon-based organic solvent may be selected from benzene, fluorobenzene, 1,2-difluorobenzene, 1,3-difluorobenzene, 1,4-difluorobenzene, 1,2,3-trifluorobenzene, 1,2,4-trifluorobenzene, chlorobenzene, 1,2-dichlorobenzene, 1,3-dichlorobenzene, 1,4-dichlorobenzene, 1,2,3-trichlorobenzene, 1,2,4-trichlorobenzene, iodobenzene, 1,2-diiodobenzene, 1,3-diiodobenzene, 1,4-diiodobenzene, 1,2,3-triiodobenzene, 1,2,4-triiodobenzene, toluene, fluorotoluene, 2,3-difluorotoluene, 2,4-difluorotoluene, 2,5-difluorotoluene, 2,3,4-trifluorotoluene, 2,3,5-trifluorotoluene, chlorotoluene, 2,3-dichlorotoluene, 2,4-dichlorotoluene, 2,5-dichlorotoluene, 2,3,4-trichlorotoluene, 2,3,5-trichlorotoluene, iodotoluene, 2,3-diiodotoluene, 2,4-diiodotoluene, 2,5-diiodotoluene, 2,3,4-triiodotoluene, 2,3,5-triiodotoluene, xylene, or combinations thereof.

The electrolyte may further include vinyl ethyl carbonate, vinylene carbonate, or an ethylene carbonate-based compound represented by Chemical Formula 3, as an additive for improving cycle life.

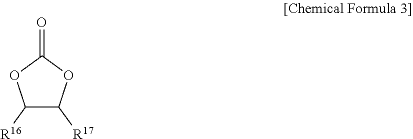

(In Chemical Formula 3, R¹⁶ and R¹⁷ are the same or different and may each independently be hydrogen, a halogen, a cyano group (CN), a nitro group (NO₂), or a C1 to C5 fluoroalkyl group, provided that at least one of R¹⁶ and R¹⁷ is a halogen, a cyano group (CN), a nitro group (NO₂), or a C1 to C5 fluoroalkyl group, and R¹⁶ and R¹¹ are not simultaneously hydrogen.)

Examples of the ethylene carbonate-based compound may be difluoro ethylene carbonate, chloroethylene carbonate, dichloroethylene carbonate, bromoethylene carbonate, dibromoethylene carbonate, nitroethylene carbonate, cyanoethylene carbonate, or fluoroethylene carbonate, and the like. An amount of the additive for improving the cycle-life characteristics may be used within an appropriate range.

The lithium salt dissolved in an organic solvent supplies a battery with lithium ions, basically operates the rechargeable lithium battery, and improves transportation of the lithium ions between a positive electrode and a negative electrode. Examples of the lithium salt include at least one or two supporting salts selected from LiPF₆, LiSbF₆, LiAsF₆, LiPO₂F₂, LiN(SO₂C₂F₅)₂, Li(CF₃SO₂)₂N, LiN(SO₃C₂F₅)₂, Li(FSO₂)₂N (lithium bis(fluorosulfonyl)imide: LiFSI), LiC₄FcSO₃, LiClO₄, LiAlO₂, LiAlCl₄, LiPO₂F₂, LiN(CₓF₂ₓ₊₁SO₂)(C_{y}F_{2y+1}SO₂), wherein x and y are natural numbers, for example, an integer of 1 to 20), lithium difluoro(bisoxolato) phosphate, LiCl, LiI, LiB(C₂O₄)₂ (lithium bis(oxalato) borate: LiBOB), and lithium difluoro(oxalato)borate (LiDFOB). A concentration of the lithium salt may range from about 0.1 M to about 2.0 M. When the lithium salt is included at the above concentration range, an electrolyte may have excellent performance and lithium ion mobility due to optimal electrolyte conductivity and viscosity.

In one embodiment, a negative electrode including the negative active material includes a negative active material layer including the negative active material and a current collector supported thereon.

The negative active material layer may include the negative active material and a binder, and further include a conductive material.

In the negative active material layer, an amount of the negative active material may be about 95 wt % to about 98 wt % based on the negative active material layer. In the negative active material layer, an amount of the binder may be about 1 wt % to about 5 wt % based on the total, 100 wt %, of the negative active material layer. Further, when the negative active material layer includes a conductive material, the negative active material layer includes about 90 wt % to about 98 wt % of the negative active material, about 1 wt % to about 5 wt % of the binder, and about 1 wt % to about 5 wt % of the conductive material.

The binder improves binding properties of negative active material particles with one another and with a current collector.

The binder includes a non-aqueous binder, an aqueous binder, or a combination thereof.

The non-aqueous binder may be an ethylene propylene copolymer, polyacrylonitrile, polystyrene, polyvinylchloride, carboxylated polyvinylchloride, polyvinylfluoride, polyurethane, polytetrafluoroethylene, polyvinylidene fluoride, polyethylene, polypropylene, polyamideimide, polyimide, or combinations thereof.

The aqueous binder may include styrene-butadiene rubber, an acrylated styrene-butadiene rubber, an acrylonitrile-butadiene rubber, an acrylic rubber, a butyl rubber, a fluorine rubber, an ethylene oxide-containing polymer, polyvinylpyrrolidone, polyepichlorohydrin, polyphosphazene, an ethylene propylene diene copolymer, polyvinylpyridine, chlorosulfonated polyethylene, latex, a polyester resin, an acrylic resin, a phenolic resin, an epoxy resin, polyvinyl alcohol, or combinations thereof.

When the aqueous binder is used as a negative electrode binder, a cellulose-based compound may be further used to provide viscosity as a thickener. The cellulose-based compound includes one or more of carboxymethyl cellulose, hydroxypropyl methyl cellulose, methyl cellulose, or alkali metal salts thereof. The alkali metal may be Na, K, or Li. The thickener may be included in an amount of about 0.1 parts by weight to about 3 parts by weight based on 100 parts by weight of the negative active material.

The conductive material is included to provide electrode conductivity, and any electrically conductive material may be used as a conductive material unless it causes a chemical change. Examples of the conductive material may be a carbon-based material such as natural graphite, artificial graphite, carbon black, acetylene black, ketjen black, a carbon fiber, and the like, a metal-based material of a metal powder or a metal fiber including copper, nickel, aluminum, silver, and the like; a conductive polymer such as a polyphenylene derivative; or a mixture thereof.

The current collector may include one selected from a copper foil, a nickel foil, a stainless steel foil, a titanium foil, a nickel foam, a copper foam, a polymer substrate coated with a conductive metal, and a combination thereof, but is not limited thereto.

In one embodiment, a positive electrode including the positive active material includes a positive active material layer including the positive active material, and a current collector supported thereon. The positive electrode active material may include lithiated intercalation compounds that reversibly intercalate and deintercalate lithium ions, and specifically, one or more composite oxides of a metal selected from cobalt, manganese, nickel, and a combination thereof, and lithium may be used. More specifically, the compounds represented by one of the following chemical formulae may be used. LiₐA_{1-b}X_{b}D₂ (0.90≤a≤1.8, 0≤b≤0.5); LiₐA_{1-b}X_{b}O_{2-c}D_{c} (0.90≤a≤1.8, 0≤b≤0.5, 0≤c≤0.05); LiₐE_{1-b}X_{b}O_{2-c}D_{c} (0.90≤a≤1.8, 0≤b≤0.5, 0≤c≤0.05); LiₐE_{2-b}X_{b}O_{4-c}D_{c} (0.90≤a≤1.8, 0≤b≤0.5, 0≤c≤0.05); LiₐN_{1-b-c}Co_{b}X_{c}D_{α} (0.90≤a≤1.8, 0≤b≤0.5, 0≤c≤0.5, 0≤α≤2); LiₐN_{1-b-c}Co_{b}X_{c}O_{2-α}T_{α} (0.90≤a≤1.8, 0≤b≤0.5, 0≤c≤0.5, 0<α<2): LiₐNi_{1-b-c}Co_{b}X_{c}O_{2-α}T₂ (0.90≤a≤1.8, 0≤b≤0.5, 0≤c≤0.5, 0<α<2); LiₐNi_{1-b-c}Mn_{b}X_{c}D_{α} (0.90≤α≤1.8, 0≤b≤0.5, 0≤c≤0.5, 0<α≤2); LiₐN_{1-b-c}Mn_{b}X_{c}O_{2-α}T_{α} (0.90≤a≤s 1.8, 0≤b≤0.5, 0≤c≤0.5, 0<α<2); LiₐNi_{1-b-c}Mn_{b}X_{c}O_{2-α}T₂ (0.90≤a≤1.8, 0≤b≤0.5, 0≤c≤0.5, 0<α<2); LiₐNi_{b}E_{c}G_{d}O₂ (0.90≤a≤1.8, 0≤b≤0.9, 0≤c≤0.5, 0.001≤d≤0.1); LiₐNi_{b}Co_{c}Mn_{d}GₑO₂ (0.90≤a≤1.8, 0≤b≤0.9, 0≤c≤0.5, 0≤d≤0.5, 0.001≤e≤0.1); LiₐNiG_{b}O₂ (0.90≤a≤1.8, 0.001≤b≤0.1) LiₐCoG_{b}O₂ (0.90≤a≤1.8, 0.001≤b≤0.1); LiₐMn_{1-b}G_{b}O₂ (0.90≤a≤1.8, 0.001≤b≤0.1); LiₐMn₂G_{b}O₄ (0.90≤a≤1.8, 0.001≤b≤0.1); LiₐMn_{1-g}G_{g}PO₄ (0.90≤a≤1.8, 0≤g≤0.5); QO₂; QS₂; LiQS₂; V₂O₅: LiV₂O₅; LiZO₂; LiNiVO₄; Li_{(3-f)}J₂ PO₄₃ (0≤f≤2); Li_{(3-f)}Fe₂ PO₄₃ (0≤f≤2); LiₐFePO₄ (0.90≤a≤1.8)

In the above chemical formulae, A is selected from Ni, Co, Mn, and a combination thereof; X is selected from Al, Ni, Co, Mn, Cr, Fe, Mg, Sr, V, a rare earth element, and a combination thereof, D is selected from O, F, S, P, and a combination thereof; E is selected from Co, Mn, and a combination thereof; T is selected from F, S, P, and a combination thereof; G is selected from Al, Cr, Mn, Fe, Mg, La, Ce, Sr, V, and a combination thereof; Q is selected from Ti, Mo, Mn, and a combination thereof; Z is selected from Cr, V, Fe, Sc, Y, and a combination thereof; and J is selected from V, Cr, Mn, Co, Ni, Cu, and a combination thereof.

Also, the compounds may have a coating layer on the surface, or may be mixed with another compound having a coating layer. The coating layer may include at least one coating element compound selected from the group consisting of an oxide of a coating element, a hydroxide of a coating element, an oxyhydroxide of a coating element, an oxycarbonate of a coating element, and a hydroxyl carbonate of a coating element. The compound for the coating layer may be amorphous or crystalline. The coating element included in the coating layer may include Mg, Al, Co, K, Na, Ca, Si, Ti, V, Sn, Ge, Ga, B, As, Zr, or a mixture thereof. The coating layer may be disposed in a method having no adverse influence on properties of a positive electrode active material by using these elements in the compound, and for example, the method may include any coating method such as spray coating, dipping, and the like, but is not illustrated in more detail since it is well-known in the related field.

The positive active material according to one embodiment may suitably be LiₐCo_{1-b}D₂ (0.90≤a≤1.8, 0≤b≤0.5), LiₐCo_{1-b}X_{b}O_{2-c}D_{c} (0.90≤a≤1.8, 0≤b≤0.5, 0≤c≤0.05); LiₐCo_{1-b}X_{b}O_{2-c}D_{c} (0≤b≤0.5, 0≤c≤0.05), or combinations thereof.

In the positive electrode, an amount of the positive active material may be about 90 wt % to about 98 wt % based on the total weight of the positive active b material layer.

In one embodiment, the positive active material layer may further include a binder and a conductive material. Herein, the amount of the binder and the conductive material may be 1 wt % to 5 wt %, respectively, based on the total amount of the positive active material layer.

The binder improves binding properties of positive electrode active material particles with one another and with a current collector, and examples of the binder may be polyvinyl alcohol, carboxymethyl cellulose, hydroxypropyl cellulose, diacetyl cellulose, polyvinylchloride, carboxylated polyvinylchloride, polyvinylfluoride, an ethylene oxide-containing polymer, polyvinylpyrrolidone, polyurethane, polytetrafluoroethylene, polyvinylidene fluoride, polyethylene, polypropylene, styrene butadiene rubber, acrylated styrene butadiene rubber, an epoxy resin, nylon, and the like, but are not limited thereto.

The conductive material is included to provide electrode conductivity, and any electrically conductive material may be used as a conductive material unless it causes a chemical change. Examples of the conductive material include a carbon-based material such as natural graphite, artificial graphite, carbon black, acetylene black, ketjen black, a carbon fiber, and the like, a metal-based material of a metal powder or a metal fiber including copper, nickel, aluminum, silver, and the like; a conductive polymer such as a polyphenylene derivative; or a mixture thereof.

The current collector may use aluminum foil, nickel foil, or a combination thereof, but is not limited thereto.

The positive active material layer and the negative active material layer may be formed by mixing an active material, a binder, and optionally a conductive material in a solvent to prepare an active material composition and coating the active material composition on a current collector. Such an active material layer preparation method is well known and thus is not described in detail in the present specification. The solvent includes N-methyl pyrrolidone and the like, but is not limited thereto. In addition, when the binder is a water-soluble binder in the negative active material layer, the solvent used for preparing the negative active material composition may be water.

Furthermore, a separator may be disposed between the positive electrode and the negative electrode depending on a type of a rechargeable lithium battery. The separator may use polyethylene, polypropylene, polyvinylidene fluoride, or multi-layers thereof having two or more layers, and may be a mixed multilayer such as a polyethylene/polypropylene double-layered separator, a polyethylene/polypropylene/polyethylene triple-layered separator, a polypropylene/polyethylene/polypropylene triple-layered separator, and the like.

According to one embodiment, the separator may also be a composite porous separator including a porous substrate and a functional layer positioned on the porous substrate. The functional layer may have additional functions, for example, may be at least one of a heat-resistance layer and an adhesive layer. The heat-resistance layer may include a heat-resistance resin and optionally a filler. In addition, the adhesive layer may include an adhesive resin and optionally a filler. The filler may be an organic filler, an inorganic filler, or combinations thereof. The heat-resistance resin and the adhesive resin may be any materials which may be used in the separator in the related art.

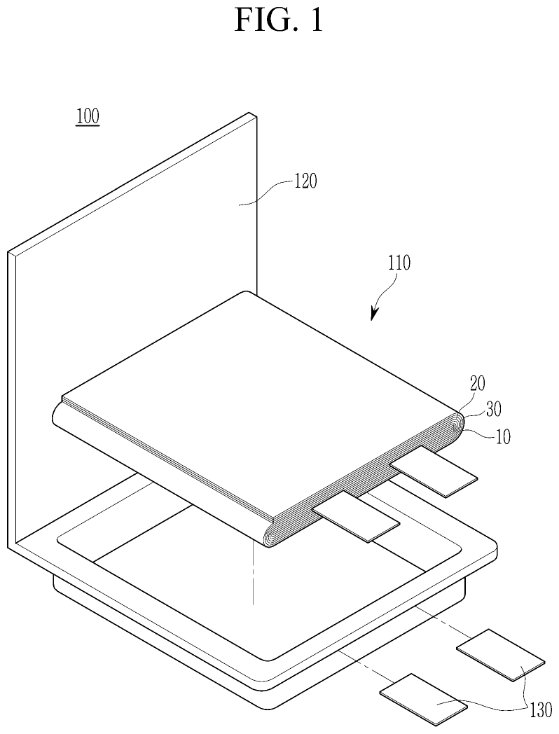

FIG. 1 is an exploded perspective view of a rechargeable lithium battery according to an embodiment of the present invention. The lithium secondary battery according to an embodiment is illustrated as a pouch battery, but is not limited thereto, and may include variously-shaped batteries such as a cylindrical battery and a prismatic pouch battery.

Referring to FIG. 1, a lithium secondary pouch battery 100 according to an embodiment includes an electrode assembly 40 manufactured by winding a positive electrode 10, a negative electrode 20, and a separator 30 disposed therebetween, a case 50 including the electrode assembly 40, and an electrode tab (130) that provides an electrical path to externally draw currents generated in the electrode assembly 40. The case 120 is sealed by overlapping the two sides facing each other. In addition, an electrolyte solution is injected into the case 120 including the electrode assembly 40 and the positive electrode 10, the negative electrode 20, and the separator 30 are impregnated in the electrolyte solution (not shown).

### Mode for Performing the Invention

Hereinafter, examples of the present invention and comparative examples are described. These examples, however, are not in any sense to be interpreted as limiting the scope of the invention.

### Example 1

1.3 M LiPF₆ was dissolved in a non-aqueous organic solvent in which ethylene carbonate, propylene carbonate, ethyl propionate, and propyl propionate were mixed in a volume % of 10:15:30:45, and a sulfolane of Chemical Formula 1a was added thereto, thereby preparing an electrolyte for a lithium secondary cell. Herein, the amount of sulfolane of Chemical Formula 1a as the first additive was set to be 2.5 wt % based on the total amount, 100 wt % of the non-aqueous organic solvent and the lithium salt.

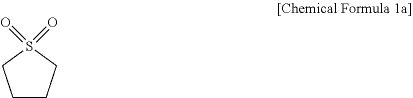

96 wt % of a negative active material in which natural graphite was mixed with the Si-carbon composite at 95:5 by weight ratio, 2 wt % of a styrene-butadiene rubber binder, and 2 wt % of carboxymethyl cellulose thickener were mixed in a water solvent to prepare a negative active material slurry. The negative active material slurry was coated on a copper foil, and dried followed by pressurizing to prepare a negative electrode. Herein, the Si-carbon composite includes a core including artificial graphite and silicon particles and a soft carbon coated on the surface of the core, and an amount of artificial graphite was 40 wt %, an amount of the silicon particles was 40 wt %, and an amount of the amorphous carbon was 20 wt % based on the total weight of the Si-carbon composite. The soft carbon coating layer had a thickness of 20 nm, and the silicon particle had an average particle diameter D50 of 100 nm.

96 wt % of a LiCoO₂ positive active material, 2 wt % of a ketjen black conductive material, and 2 wt % of polyvinylidene fluoride were mixed in an N-methyl pyrrolidone solvent to prepare a positive active material slurry. The positive active material slurry was coated on an aluminum foil and dried followed by pressurizing to prepare a positive electrode.

Using the electrolyte, the positive electrode, and the negative electrode, a 4.4 V grade pouch lithium secondary cell was fabricated according to the convention procedure.

### Example 2

An electrolyte was prepared by the same procedure as in Example 1, except that the amount of the additive of Chemical Formula 1a was changed to 5 wt % based on the total amount, 100 wt %, of the non-aqueous organic solvent and the lithium salt, and a pouch-type lithium secondary cell was fabricated by the same procedure as in Example 1, except that the electrolyte, and the negative electrode and the positive electrode of Example 1 were used.

### Example 3

An electrolyte was prepared by the same procedure as in Example 1, except that the amount of the additive of Chemical Formula 1a was changed to 10 wt % based on the total amount, 100 wt % of the non-aqueous organic solvent and the lithium salt, and a pouch-type lithium secondary cell was fabricated by the same procedure as in Example 1, except that the electrolyte, and the negative electrode and the positive electrode of Example 1 were used.

### Reference Example 1

A negative electrode was prepared by the same procedure as in Example 1, except that a mixing ratio of natural graphite and the Si-carbon composite was changed to 95:5 by weight ratio, an electrolyte was prepared by the same procedure as in Example 1, except that the amount of the additive of Chemical Formula 1a was changed to 12.5 wt % based on the total amount, 100 wt % of the non-aqueous organic solvent and the lithium salt, and a pouch-type lithium secondary cell was fabricated by the same procedure as in Example 1, except that the electrolyte, and the negative electrode and the positive electrode of Example 1 were used.

### Example 4

A negative electrode was prepared by the same procedure as in Example 1, except that a mixing ratio of natural graphite and the Si-carbon composite was changed to 97.5:2.5 by weight ratio, a pouch-type lithium secondary cell was fabricated by the same procedure as in Example 4, except that the electrolyte, and the negative electrode and the positive electrode of Example 1 were used.

### Example 5

A pouch-type lithium secondary cell was fabricated by the same procedure as in Example 4, except that the negative electrode of Example 4, the electrolyte of Example 2, and the positive electrode of Example 1 were used.

### Example 6

A pouch-type lithium secondary cell was fabricated by the same procedure as in Example 4, except that the negative electrode of Example 4, the electrolyte of Example 3, and the positive electrode of Example 4 were used.

### Reference Example 2

A pouch-type lithium secondary cell was fabricated by the same procedure as in Example 4, except that the negative electrode of Example 4, the electrolyte of Reference Example 1, and the positive electrode of Example 4 were used.

### Comparative Example 1

1.3 M LiPF₆ was dissolved in a non-aqueous organic solvent in which ethylene carbonate, propylene carbonate, ethyl propionate, and propyl propionate were mixed in a volume % of 10:1530:45 to prepare an electrolyte for a lithium secondary cell.

96 wt % of a natural graphite negative active material, 2 wt % of a styrene-butadiene rubber binder, and 2 wt % of carboxymethyl cellulose thickener were mixed in a water solvent to prepare a negative active material slurry. The negative active material slurry was coated on a copper foil, and dried followed by pressurizing to prepare a negative electrode.

A pouch-type lithium secondary cell was fabricated by the same procedure as in Example 1, except that the electrolyte, and the negative electrode and the positive electrode of Example 1 were used.

### Comparative Example 2

A pouch-type lithium secondary cell was fabricated by the same procedure as in Comparative Example 1, except that the electrolyte of Example 1, the negative electrode of Comparative Example 1, and the positive electrode of Comparative Example 1 were used.

### Comparative Example 3

A pouch-type lithium secondary battery was prepared by the same procedure as in Comparative Example 2, except that an electrolyte prepared by changing the amount of the sulfolane of Chemical Formula 1a to 5 wt % based on the total amount, 100 wt % of the non-aqueous organic solvent and the lithium salt, was used.

### Comparative Example 4

A pouch-type lithium secondary battery was prepared by the same procedure as in Comparative Example 2, except that an electrolyte prepared by changing the amount of the sulfolane of Chemical Formula 1a to 10 wt % based on the total amount, 100 wt % of the non-aqueous organic solvent and the lithium salt, was used.

### Comparative Example 5

A pouch-type lithium secondary cell was fabricated by the same procedure as in Comparative Example 1, except that the electrolyte of Comparative Example 1, the negative electrode of Example 1, and the positive electrode of Comparative Example 1 were used.

### Comparative Example 6

A pouch-type lithium secondary cell was fabricated by the same procedure as in Comparative Example 1, except that the electrolyte of Comparative Example 1, the negative electrode of Example 5, and the positive electrode of Comparative Example 1 were used.

### Comparative Example 7

A negative electrode was prepared by the same procedure as in Example 1, except that a mixing ratio of natural graphite and the Si-carbon composite was changed to 92.5:7.5 by weight ratio, and a pouch-type lithium secondary cell was fabricated by the same procedure as in Comparative Example 1, except that the negative electrode, the electrolyte of Comparative Example 3, and the positive electrode of Comparative Example 1 were used.

The mixing ratio and the amount of sulfolane represented by Chemical Formula 1a of Examples 1 to 6, Reference Examples 1 and 2, and Comparative Examples 1 to 7 are summarized in Table 1.

TABLE 1

| | | | Amount of sulfolane |
|---|---|---|---|
| | Graphite | Si-carbon composite | of Chemical |
| | (wt %) | (wt %) | Formula 1a (wt %) |
| Comparative | 100 | — | 0 |
| Example 1 | | | |
| Comparative | 100 | — | 2.5 |
| Example 2 | | | |
| Comparative | 100 | — | 5 |
| Example 3 | | | |
| Comparative | 100 | — | 10 |
| Example 4 | | | |
| Comparative | 95 | 5 | 0 |
| Example 5 | | | |
| Comparative | 97.5 | 2.5 | 0 |
| Example 6 | | | |
| Comparative | 92.5 | 7.5 | 5 |
| Example 7 | | | |
| Example 1 | 95 | 5 | 2.5 |
| Example 2 | 95 | 5 | 5 |
| Example 3 | 95 | 5 | 10 |
| Reference | 95 | 5 | 12.5 |
| Example 1 | | | |
| Example 4 | 97.5 | 2.5 | 2.5 |
| Example 5 | 97.5 | 2.5 | 5 |
| Example 6 | 97.5 | 2.5 | 10 |
| Reference | 97.5 | 2.5 | 12.5 |
| Example 2 | | | |

* Evaluation of DC internal resistance (DC-IR: Direct current internal resistance)

The lithium secondary cells according to Examples 1 to 6, Reference Examples 1 and 2, and Comparative Examples 1 to 7 were constant-discharged at 10 A for 10 seconds under the SOC100 (state of charge, fully charged state, charged to be 100% of charge capacity based 100% of entire battery charge capacity) at 60° C., constant-discharged at 10 A for 10 seconds, constant-discharged at 1 A for 10 seconds, and constant-discharged at 10 A for 4 seconds, a voltage value and a current value were measured right before storage, and furthermore, the cell was stored at 60° C. for 30 days, and then a voltage value and a current value were measured.

The DC resistance (DC-IR) was calculated from the data at 18 seconds and 23 seconds by the equation ΔR=ΔV/ΔI. That is, it was obtained from (voltage measured after 10 A for 10 seconds discharge, 1 A for 10 seconds discharge, and 10 A for 4 seconds discharge-voltage measured after 10 A for 10 seconds discharge and 1 A for 8 seconds discharge)/current after 10 A for 10 seconds discharge and 8 seconds discharge.

The DCIR resistance increase rate was calculated from the DC resistance just before storage and the DC resistance after 30 days by Equation 1.

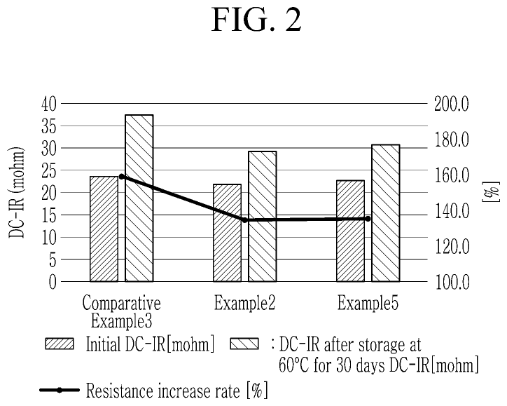

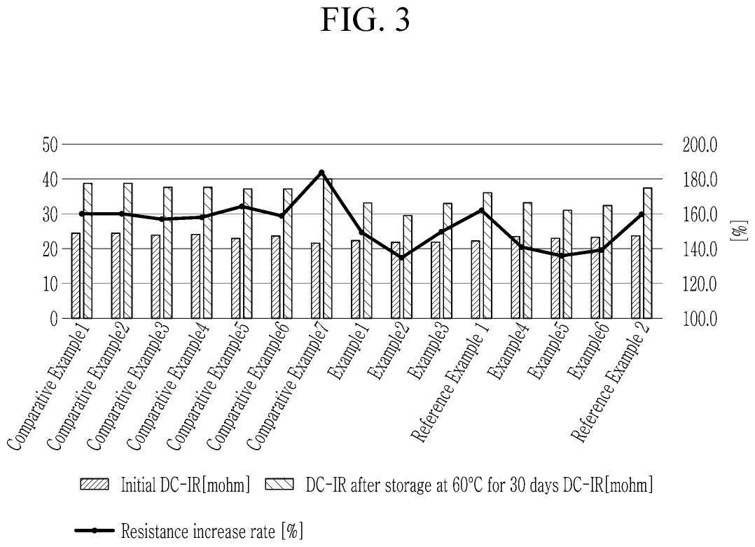

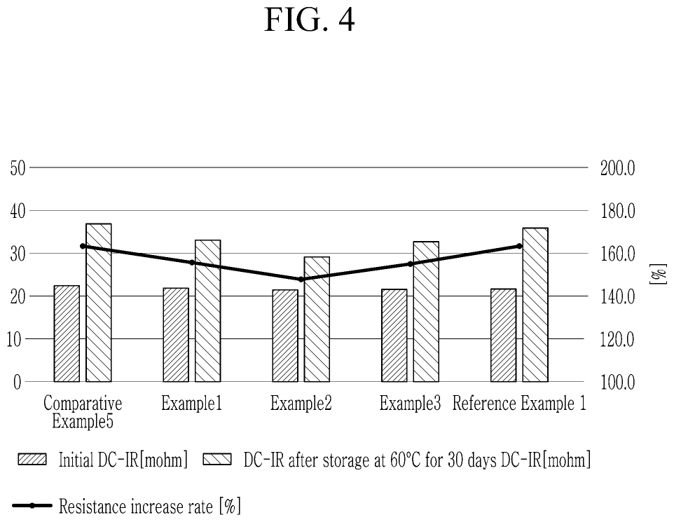

As results, the initial DC-IR and resistance increase rate and DC-IR after 3 days at 60° C. are shown in Table 1. In addition, in order clearly confirm the effects depending on the amounts of the Si-carbon composite, the results of Examples 2 and 5 and Comparative Example 3 are shown in FIG. 2, and the results of Examples 1 to 6, Reference Examples 1 and 2, and Comparative Examples 1 to 7 are shown in FIG. 3. Furthermore, in order to clearly identify the effects depending on the amounts of sulfolane of Chemical Formula 1a, the results are shown in FIG. 4.

DCIR increase rate=[DCIR 30 d.]/DCIR (0 d.)×100% [Equation 1]

In Equation 1, DCIR 30 d. indicates DCIR after 30 days, and DCIR (0 d.) indicates DCIR just before storage.

TABLE 2

| | | DC-IR after storage | |
|---|---|---|---|
| | Initial DC-IR | at 60° C. for | Resistance |
| | (mohm) | 30 days (mohm) | increase rate (%) |
| Comparative | 24.3 | 38.8 | 159.7 |
| Example 1 | | | |
| Comparative | 24.2 | 38.6 | 159.5 |
| Example 2 | | | |
| Comparative | 23.8 | 37.5 | 157.6 |
| Example 3 | | | |
| Comparative | 23.9 | 37.8 | 158.2 |
| Example 4 | | | |
| Comparative | 22.5 | 36.9 | 164.0 |
| Example 5 | | | |
| Comparative | 23.4 | 37.2 | 159.0 |
| Example 6 | | | |
| Comparative | 21.4 | 40.1 | 187.4 |
| Example 7 | | | |
| Example 1 | 22.2 | 33.1 | 149.1 |
| Example 2 | 21.8 | 29.4 | 134.9 |
| Example 3 | 21.9 | 32.8 | 149.8 |
| Reference | 22.1 | 35.8 | 162.0 |
| Example 1 | | | |
| Example 4 | 23.5 | 33.1 | 140.9 |
| Example 5 | 22.8 | 30.9 | 135.5 |
| Example 6 | 23.2 | 32.3 | 139.2 |
| Reference | 23.3 | 37.2 | 159.7 |
| Example 2 | | | |

As shown in Table 2 and FIG. 3, the lithium secondary cells according to Examples 1 to 6 in which artificial graphite and the Si-carbon composite were used as the negative active material and the electrolyte using the sulfolane of Chemical Formula 1a, particularly, at an amount of 0.1 wt % to 10 wt % was used, exhibited a low resistance increase rate after storage at high temperature, while the suitable initial resistance was maintained.

Although artificial graphite and Si-carbon composite were used as the negative electrode and the sulfolane of Chemical Formula 1a, Reference Examples 1 and 2 using a large amount of 12.5 wt % of sulfolane exhibited a much higher resistance increase rate after storage at high temperature.

As the results Comparative Examples 1 to 7 shown in Table 2 and FIG. 3, when the Si-carbon composite is not used as the negative active material, even if the sulfolane of Chemical Formula 1a was included, the resistance increase rate at high temperature highly exhibited. From the results shown in FIG. 2, it can be clearly shown that the effects for reducing the resistance increase rate at high temperature storage and DC-IR after storage at 60° C. for 30 days by adding sulfolane of Chemical Formula 1a were obtained when the Si-carbon composite was included as the negative active material.

In addition, it can be clearly shown from the results of FIG. 4 that using the electrolyte with sulfolane of Chemical Formula 1a at an amount of 2.5 wt %, 5 wt %, and 10 wt %, respectively renders to decrease the resistance increase rate at high temperature storage and DC-IR after storage at 60° C. for 30 days.

While this invention has been described in connection with what is presently considered to be practical example embodiments, it is to be understood that the invention is not limited to the disclosed embodiments, but, on the contrary, is intended to cover various modifications and equivalent arrangements included within the spirit and scope of the appended claims. Therefore, the aforementioned embodiments should be understood to be examples but not limiting the present invention in any way.

1. A lithium secondary battery, comprising:
an electrolyte comprising a non-aqueous organic solvent, a lithium salt, and an additive represented by Chemical Formula 1;
a negative electrode comprising a negative active material comprising a Si-carbon composite; and
a positive electrode comprising a positive active material: 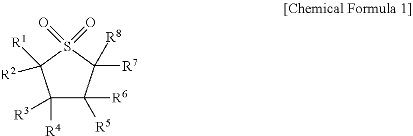
(in Chemical Formula 1,
R¹ to R⁸ are each independently a hydrogen atom, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C3 to C30 cycloalkynyl group, or a substituted or unsubstituted C6 to C30 aryl group.)

2. The lithium secondary battery of claim 1, wherein the R¹ to R⁸ are each independently a hydrogen atom, a substituted or unsubstituted C1 to C10 alkyl group, a substituted or unsubstituted C2 to C10 alkenyl group, a substituted or unsubstituted C2 to C10 alkynyl group, a substituted or unsubstituted C3 to C10 cycloalkyl group, a substituted or unsubstituted C3 to C10 cycloalkenyl group, a substituted or unsubstituted C3 to C10 cycloalkynyl group, or a substituted or unsubstituted C6 to C10 aryl group.

3. The lithium secondary battery of claim 1, wherein the additive represented by Chemical Formula 1 includes sulfolane, methylsulfolane, dimethylsulfolane, or combinations thereof.

4. The lithium secondary battery of claim 1, wherein an amount of the additive represented by Chemical Formula 1 is 0.1 wt % to 10 wt % when amounts of the non-aqueous organic solvent and the lithium salt are to be 100 wt %.

5. The lithium secondary battery of claim 1, wherein an amount of the Si—C carbon composite is 0.1 wt % to 5 wt % based on the total weight of the negative active material.

6. The lithium secondary battery of claim 1, wherein the negative active material further comprises crystalline carbon.

7. The lithium secondary battery of claim 1, wherein the non-aqueous organic solvent includes a propionate-based solvent.

8. The lithium secondary battery of claim 7, wherein the propionate-based solvent is methyl propionate, ethyl propionate, propyl propionate, or combinations thereof.

9. The lithium secondary battery of claim 7, wherein an amount of the propionate-based solvent is 5 volume % to 80 volume % based on the total volume of the non-aqueous organic solvent.

10. The lithium secondary battery of claim 1, wherein the Si-carbon composite comprises Si nanoparticles and amorphous carbon.

11. The lithium secondary battery of claim 1, wherein the Si-carbon composite comprises a core and a coating layer surrounded on the core,
the core comprises amorphous carbon or crystalline carbon, and Si nanoparticles, and
the coating layer comprises amorphous carbon.

12. The lithium secondary battery of claim 11, wherein the coating layer has a thickness of 1 nm to 100 nm.

13. The lithium secondary battery of claim 11, wherein an amount of the Si nanoparticles is 1 wt % to 60 wt % based on the total 100 wt % of the Si-carbon composite.
